(19) United States
(12) Patent Application Publication
(10) Pub. No.: US 2026/0191745 A1
(43) Pub. Date: Jul. 9, 2026

(54) Pre-Soaked Stain-Removal Wipe Pad for Oral Surfaces

(71) Applicant: Sandy Warren Bubalo, Pasadena, CA (US)

(72) Inventor: Sandy Warren Bubalo, Pasadena, CA (US)

(21) Appl. No.: 19/407,026

(22) Filed: Dec. 3, 2025

Related U.S. Application Data
(60) Provisional application No. 63/742,306, filed on Jan. 6, 2025.

(51) Int. Cl. A61K 8/02; A61Q 11/00

(52) U.S. Cl. CPC A61K 8/0208; A61Q 11/00; A61K 2800/28; A61K 2800/92

(57) ABSTRACT

An oral care wipe device for removing red wine stains from lips, teeth, and oral tissues is disclosed. The device includes a flexible, absorbent, and biocompatible nonwoven wipe pad having a generally semicircular body defined by a convex outer edge and a bow-shaped inner edge with a central dip and opposing peak members. An aperture at a longitudinal end provides access to a tubular slit configured to receive and retain a fingertip, enabling the wipe pad to be worn as a finger sleeve. The wipe pad has a textured exterior surface, and the pad is pre-soaked in a cleaning solution comprising vinegar, baking soda, a natural flavoring agent, soda water, and purified water. In use, the pad provides frictional scrubbing while delivering the solution to dissolve stains.

### CROSS-REFERENCE TO RELATED APPLICATION

The present application claims priority to, and the benefit of, U.S. Provisional Application No. 63/742,306 which was filed on Jan. 6, 2025 and is incorporated herein by reference in its entirety.

### FIELD OF THE INVENTION

The present invention generally relates to oral care devices for cleaning and stain removal. More specifically, the present invention relates to a stain-removal pad device. The invention comprises a wipe pad formed of a flexible, absorbent, and biocompatible nonwoven material such as cotton fibers, biodegradable cellulose fibers, or composites thereof. The wipe pad has a lip-conforming geometry. An aperture is disposed at one longitudinal end of the wipe pad; the aperture defines a tubular slit dimensioned to receive a fingertip of a user so that the wipe pad may be worn in a finger-sleeve configuration. A textured exterior surface is provided to increase friction for scrubbing oral surfaces, while the pad is impregnated with a natural cleaning solution containing vinegar, baking soda, soda water, and a flavoring agent. In use, the device enables the user to scrub stained lips, teeth, and oral tissue with precision and control, Accordingly, the present disclosure makes specific reference thereto. Nonetheless, it is to be appreciated that aspects of the present invention are also equally applicable to other like applications, devices, and methods of manufacture.

### BACKGROUND

By way of background, many individuals like consuming red wine frequently. Such individuals encounter the problem of temporary but noticeable stains forming on the lips, teeth, and tongue. The stains are caused by pigments such as tannins and anthocyanins naturally present in red wine, which adhere to oral surfaces and create an unsightly appearance. For some individuals, the prospect of developing such stains is discouraging and may reduce their enjoyment of red wine altogether.

Conventional methods of addressing this problem generally require immediate oral hygiene practices, such as brushing the teeth or rinsing the mouth shortly after wine consumption. However, the conventional approaches are often impractical, inconvenient, and socially disruptive, particularly in public settings such as restaurants, bars, or social gatherings. Accordingly, individuals desire a compact and convenient solution that enables individuals to quickly and effectively remove red wine stains from the mouth area without the necessity of full oral hygiene procedures.

Therefore, there exists a long-felt need in the art for an oral care device that eliminates the shortcomings of conventional solutions for red wine stain removal. There is a need for a compact and convenient stain-removal apparatus that may be discreetly carried and used in social settings without requiring access to toothbrushes, toothpaste, or mouthwash. Further, there is a need in the art for an oral care wipe that provides effective stain removal from lips, teeth, and surrounding oral tissue immediately after consumption of red wine or other staining agents such as coffee, tea, or berries. Additionally, there is a need for a device that combines mechanical scrubbing with a natural cleaning solution to deliver effective results without harsh chemicals or alcohol. Finally, there exists a need for an oral care product that is disposable, hygienic, and portable, and offers a way to quickly remove red wine stains.

The subject matter disclosed and claimed herein, in one embodiment, comprises a stain-removal pad device featuring a pre-moistened, semi-circular wipe pad configured for oral use. The device includes a wipe pad formed of a flexible, absorbent, and biocompatible nonwoven material such as cotton fibers, biodegradable cellulose fibers, or composites thereof. The wipe pad is defined by a convex outer edge and a bow-shaped inner edge with a central dip and opposing peak members to provide a lip-conforming geometry. An aperture is disposed at one longitudinal end of the pad, the aperture opening into a tubular slit dimensioned to receive a fingertip of a user such that the pad may be worn securely in a finger-sleeve configuration. A textured exterior surface is provided to increase friction and enhance scrubbing against stained oral surfaces, and the pad is pre-soaked in a cleaning solution comprising vinegar, baking soda, a natural flavoring or scenting agent, soda water, and purified water.

In one embodiment, the wipe pad is individually packaged in sterile packets or stored in a lip-shaped container having a base portion and lid portion joined by a hinge, snap-fit, or frictional engagement to maintain moisture and sterility until use.

In this manner, the stain-removal pad device of the present invention addresses longstanding shortcomings of conventional oral hygiene methods by enabling an immediate and effective stain removal solution. The device enables users to remove or reduce visible red wine stains on lips, teeth, and oral tissue in social environments without the inconvenience of brushing or rinsing. The device can be easily used using the finger-sleeve configuration and is further pre-soaked natural cleaning solution for enabling a hygienic disposable product.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an oral care wipe device. The device comprises a wipe pad that is formed of a flexible, absorbent, and biocompatible nonwoven material such as cotton fibers, biodegradable cellulose fibers, or a composite thereof. The wipe pad has a generally semicircular body defined by a convex outer edge and a bow-shaped inner edge, with the bow-shaped inner edge including a central dip and two peak members positioned on opposing sides of the dip to provide a lip-conforming shape. An aperture is disposed at a first longitudinal end of the wipe pad, that provides access to a tubular slit dimensioned to receive and retain a fingertip of a user, thereby enabling the wipe pad to be worn as a finger sleeve. The exterior surface of the wipe pad is textured to increase friction and to provide gentle scrubbing action against stained oral surfaces.

In another embodiment, a stain-removal pad device for oral care is disclosed. The pad device comprises a wipe pad formed of a flexible, absorbent, and biocompatible nonwoven material selected from the group consisting of cotton fibers, biodegradable cellulose fibers, and composites thereof. The wipe pad has a generally semicircular body defined by a convex outer edge and a bow-shaped inner edge, the bow-shaped inner edge including a central dip and two peak members disposed on opposing sides of the central dip to provide a lip-conforming shape. An aperture is disposed at a first longitudinal end of the wipe pad, the aperture provides access to a tubular slit dimensioned to receive and retain a fingertip of a user so that the wipe pad is wearable as a finger sleeve. The wipe pad further includes a textured exterior surface configured to provide enhanced friction. The wipe pad is impregnated with a liquid cleaning solution that includes vinegar in an amount between about 1% and 5% by weight, baking soda in an amount between about 0.5% and 3% by weight, a natural flavoring agent in an amount between about 0.05% and 0.2% by weight, soda water in an amount between about 0.1% and 0.5% by weight, and purified water as a solvent.

In use, when the fingertip of the user is inserted into the aperture, the wipe pad is adapted to be manipulated against stained oral surfaces including lips and teeth such that the textured exterior surface provides frictional scrubbing while simultaneously delivering the liquid cleaning solution to dissolve and remove red wine stains.

In one embodiment, a method of removing red wine stains from an oral surface of a user is described and comprises providing an oral care wipe device that has a wipe pad with an aperture and a tubular slit configured to receive a fingertip of the user. The user inserts the fingertip into the aperture to form a finger-sleeve configuration of the wipe pad. The method further includes contacting a textured exterior surface of the wipe pad, which has been pre-soaked in a cleaning solution, against a stained oral surface such as lips, teeth, or surrounding oral tissue. The wipe pad is then rubbed against the stained oral surface to provide frictional scrubbing while simultaneously delivering the cleaning solution, thereby removing or reducing visible red wine stains.

In yet another embodiment, the oral care wipes are stored in a storage container that comprises a base portion and a lid portion, each shaped to resemble human lips. The base portion defines an interior cavity that is dimensioned to receive and retain a plurality of semicircular wipe pads. The lid portion is configured to mate with and cover the base portion to enclose the interior cavity, thereby preventing exposure of the stored wipe pads to the external atmosphere.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of oral care wipe device of the preset invention in accordance with the disclosed structure;

FIG. 2 illustrates a block diagram showing ingredients of the liquid cleaning solution in which the oral care wipe device is presoaked in accordance with the disclosed structure;

FIG. 3 illustrates a perspective view of the stain-removal wipe pad device showing the aperture to enable the device to be used in a finger-sleeve configuration in accordance with the disclosed structure;

FIG. 4 illustrates a perspective view of the stain-removal wipe pad of the present invention being held by a user's hand in a ready-to-use configuration in accordance with the disclosed structure;

FIG. 5 illustrates a perspective view of a user employing the stain-removal pad device of the present invention in a cleaning operation against the stained oral surface (i.e., lips and teeth) in accordance with the disclosed structure; and

FIG. 6 illustrates a perspective view of a lip-shaped container for storing one or more stain-removal pad devices in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an oral care device that eliminates the shortcomings of conventional solutions for red wine stain removal. There is a need for a compact and convenient stain-removal apparatus that may be discreetly carried and used in social settings without requiring access to toothbrushes, toothpaste, or mouthwash. Further, there is a need in the art for an oral care wipe that provides effective stain removal from lips, teeth, and surrounding oral tissue immediately after consumption of red wine or other staining agents such as coffee, tea, or berries. Additionally, there is a need for a device that combines mechanical scrubbing with a natural cleaning solution to deliver effective results without harsh chemicals or alcohol. Finally, there exists a need for an oral care product that is disposable, hygienic, and portable, and offers a way to quickly remove red wine stains.

The present invention, in one exemplary embodiment, is a stain removal wipe pad device that is formed of a flexible, absorbent, and biocompatible nonwoven material such as cotton fibers, biodegradable cellulose fibers, or a composite thereof. The wipe pad has a generally semicircular body defined by a convex outer edge and a bow-shaped inner edge, with the bow-shaped inner edge including a central dip and two peak members positioned on opposing sides of the dip to provide a lip-conforming shape. An aperture is disposed at a first longitudinal end of the wipe pad, that provides access to a tubular slit dimensioned to receive and retain a fingertip of a user, thereby enabling the wipe pad to be worn as a finger sleeve. The exterior surface of the wipe pad is textured to increase friction and to provide gentle scrubbing action against stained oral surfaces.

Reference will now be made in detail to the present preferred embodiments of the invention, examples of which are illustrated in the accompanying drawings. Wherever possible, the same reference numerals are used in the drawings and the description to refer to the same or like parts.

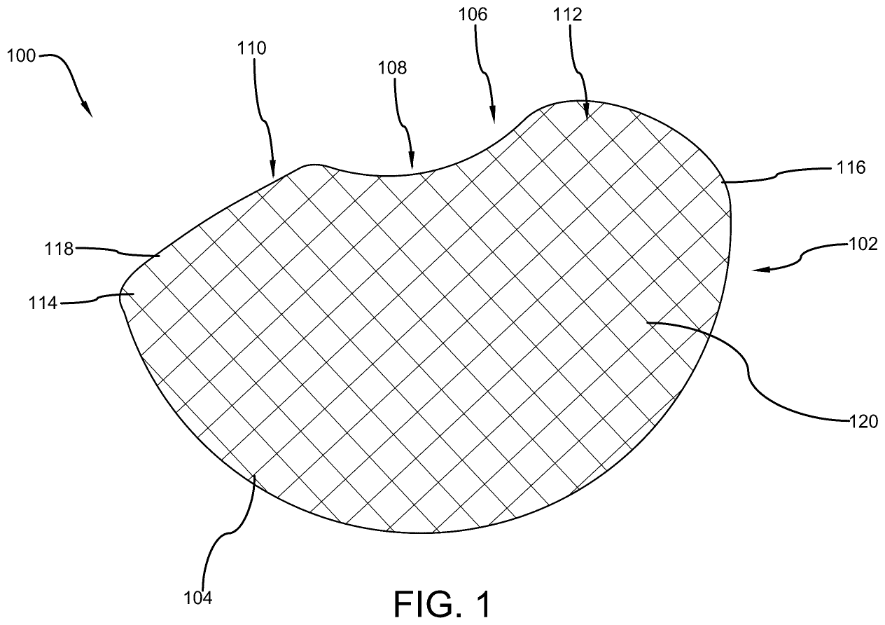

Referring initially to the drawings, FIG. 1 illustrates a perspective view of oral care wipe device of the preset invention in accordance with the disclosed structure. The oral care wipe device 100 of the present invention is designed as a compact and convenient oral care wipe specifically designed to remove red wine stains from lips, teeth, and the mouth area. The oral care wipe device 100 includes a pre-moistened wipe pad 102 formed of a flexible, absorbent, and biocompatible nonwoven material such as cotton fibers, biodegradable cellulose fibers, or a composite thereof. The wipe pad 102 has a generally semicircular or arcuate body configured to conform to lips and/or teeth of a user. More specifically, the wipe pad 102 has a convex outer edge 104 and a bow-shaped inner edge 106 for providing an arcuate human lip-shaped body to the wipe pad 102. The bow-shaped inner edge 106 has a central dip 108 and two peak members 110, 112 extending on opposing sides of the central dip 108.

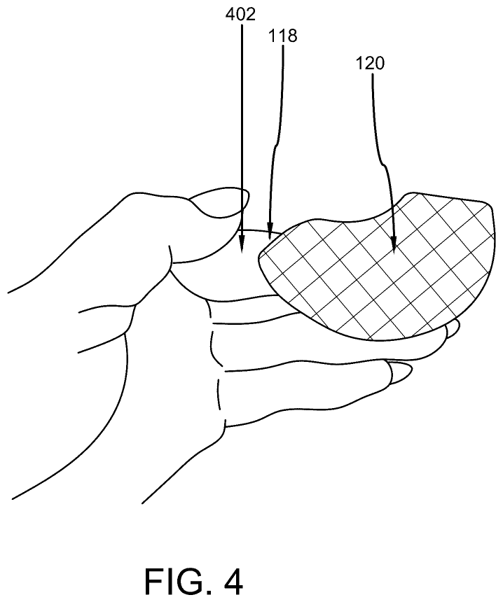

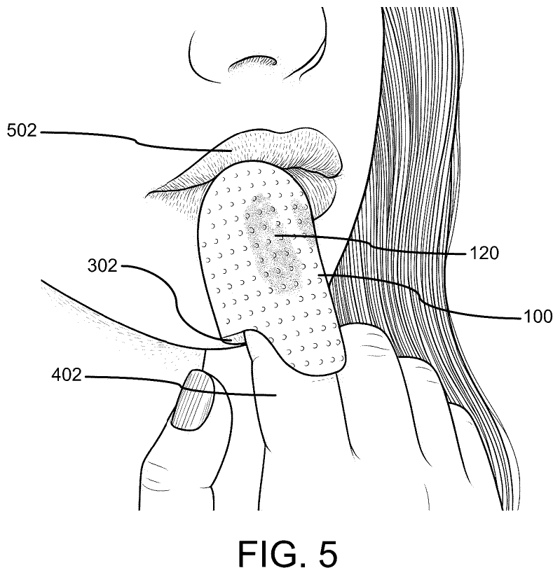

The wipe pad 102 has a length extending between the longitudinal terminal ends 114, 116 in the range from about 3 cm to about 8 cm. In the preferred embodiment, the oral care wipe device 100 is pre-soaked with a liquid cleaning solution for helping remove visible red wine stains from lips, teeth, and surrounding oral tissues when the device 100 is rubbed against stained oral surfaces. The wipe pad 102 has an aperture 118 at the first end 114 (originating from the inner edge 106) for enabling the device 100 to be used as a finger-sleeve wherein the device 100 is adapted to receive and be retained upon a fingertip of a user for use as illustrated in FIGS. 4 and 5. The exterior surface 120 of the wipe pad 102 is textured for increasing friction for gentle scrubbing against stained surfaces. In the preferred embodiment, the wipe pad is designed as disposable after a single use, thereby preventing cross-contamination or re-use degradation.

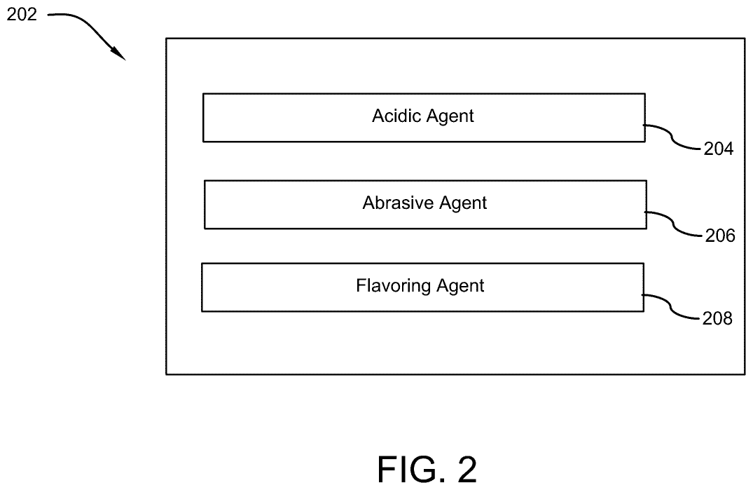

FIG. 2 illustrates a block diagram showing ingredients of the liquid cleaning solution in which the oral care wipe device is presoaked in accordance with the disclosed structure. The solution or compound 202 includes an acidic agent 204 such as vinegar for effectively dissolving wine tannins and pigments. An alkaline or mildly abrasive agent 206 such as baking soda is included for stain removal. A natural flavoring or scenting agent 208 such as spearmint oil, peppermint oil, citrus extract, or menthol is included in the solution or compound 202 and imparts oral freshness and is configured to impart a fresh breath sensation that persists for at least 10 minutes post-application. The cleaning solution or compound 202 is free of alcohol, synthetic dyes, or harsh chemical whitening agents. The cleaning solution or compound 202 enables the device 100 to be adapted for use after consumption of other staining agents, including coffee, tea, and berries. A solvent 210 such as water carries all the ingredients and can be distilled or purified water.

TABLE 1

| Ingredients | Solution in percentage (% w/v) |
|---|---|
| Vinegar | 1-5% |
| Baking Soda | 0.5-3% |
| Spearmint Oil or Peppermint Oil | 0.05-0.2% |
| Soda water | 0.1-0.5% |
| Solvent (Water) | Up to 100% |

In some embodiments, a natural preservative such as citric acid can also be included in the solution for preservation of the device 100 and for adjusting the pH from 4.5 to 5.5 (i.e., adding citric acid in an amount sufficient to adjust the pH to a value of from 4.5 to 5.5).

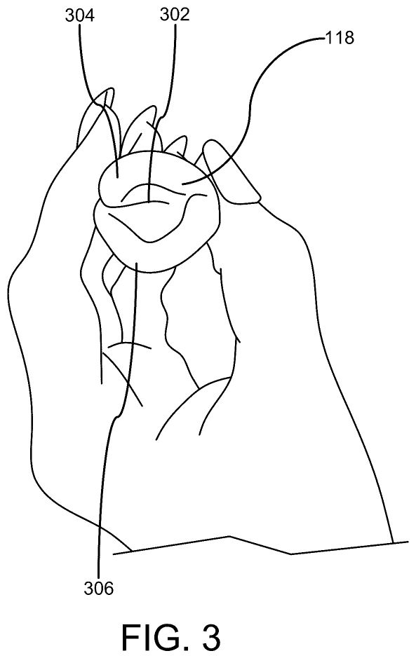

FIG. 3 illustrates a perspective view of the stain-removal wipe pad device showing the aperture to enable the device to be used in a finger-sleeve configuration in accordance with the disclosed structure. The aperture 118 disposed in the wipe pad device 100 provides access to a tubular slit 302 which is dimensioned to receive a fingertip of the user. The wipe pad 102 is flexible and conformable, thereby creating the secure finger sleeve. A fingertip of user, when inserted into the aperture 118, provides firm support and control, enabling the wipe pad 100 to be manipulated with precision against stained surfaces. The slit 302 forms the two opposite sides 304, 306 of the wipe pad 102, wherein both the sides 304, 306 overlap or contact each other.

FIG. 4 illustrates a perspective view of the stain-removal wipe pad of the present invention being held by a user's hand in a ready-to-use configuration in accordance with the disclosed structure. When the stain-removal wipe pad device 100 is equipped or mounted on a fingertip 402 and is in a flat state, the textured exterior surface 120 is accessible and is configured to provide enhanced friction for mechanical stain removal. The user may grasp the pad device 100 and subsequently manipulate the pad device 100 to access the tubular slit 302, as described in FIG. 3. The pad device 100 can be positioned for direct wiping across the lips and teeth and applied directly against oral surfaces, enabling the cleaning solution or compound 202 impregnated within the pad device 100 to interact with red wine stains while simultaneously benefiting from the gentle scrubbing action of the textured surface.

FIG. 5 illustrates a perspective view of a user employing the stain-removal pad device of the present invention in a cleaning operation against the stained oral surface (i.e., lips and teeth) in accordance with the disclosed structure. In use, the pad device 100 is securely retained or mounted on the fingertip 402 using the slit 302 which enables the user to press and wipe the textured cleaning surface 120 of the pad against the stained oral surface 502 for frictional scrubbing for cleaning while simultaneously delivering a pre-impregnated cleaning solution to the stained oral area 502.

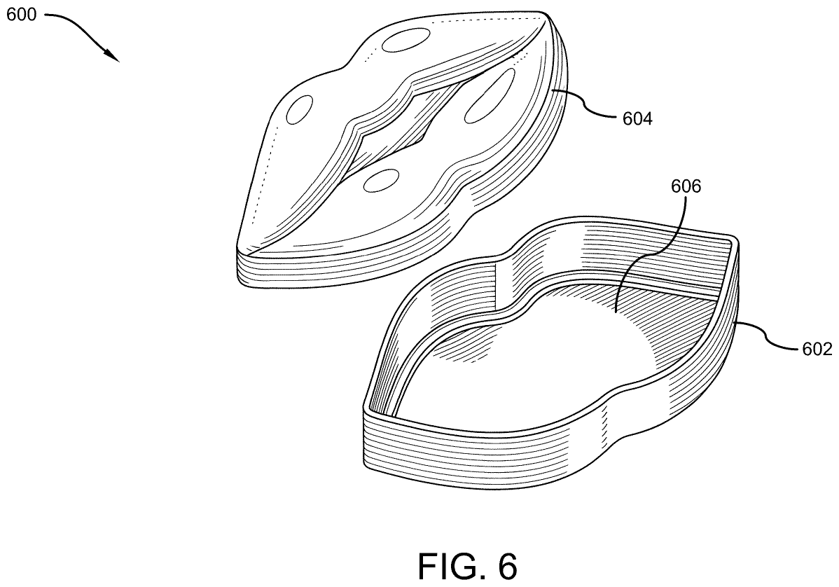

FIG. 6 illustrates a perspective view of a lip-shaped container 600 for storing one or more stain-removal pad devices in accordance with the disclosed structure. In the present embodiment, the container 600 includes a base portion 602 and a lid portion 604. The two portions 602, 604 are designed to resemble human lips and conform to the shape of the stain-removal pad device of the present invention. The lid portion 604 is configured to mate with and cover the base portion 602, thereby enclosing the interior cavity 606 and preventing the exposure of the stored stain-removal pad devices from the external atmosphere.

The interior cavity 606 is dimensioned to receive and retain a plurality of the semi-circular pad devices. The lip-shaped exterior provides a compact and portable storage solution, sized for carriage within a pocket, purse, or handbag. It should be noted that the lid portion 604 may be coupled to the base portion 602 by a hinge, snap-fit, or frictional engagement, thereby enabling repeated opening and closing while maintaining a sealed environment to preserve the moisture content of the pre-impregnated pads. Each wipe pad can also be contained within an individually sealed sterile packet to maintain moisture and freshness prior to use.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “oral care wipe device”, “stain-removal pad device”, “stain-removal wipe pad”, and “wipe pad” are interchangeable and refer to the stain-removal wipe pad for oral surfaces 100 of the present invention.

Notwithstanding the forgoing, the stain-removal wipe pad for oral surfaces 100 of the present invention can be of any suitable configuration as is known in the art without affecting the overall concept of the invention, provided that it accomplishes the above stated objectives. One of ordinary skill in the art will appreciate that the stain-removal wipe pad for oral surfaces 100 shown in the FIGS. are for illustrative purposes only, and that many other configurations of the stain-removal wipe pad for oral surfaces 100 are well within the scope of the present disclosure. Although the dimensions of the stain-removal wipe pad for oral surfaces 100 are important design parameters for user convenience, the stain-removal wipe pad for oral surfaces 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An oral care device comprising:
a wipe pad; and
a liquid solution;
wherein said wipe pad comprising an absorbent material selected from the group consisting of cotton fibers, biodegradable cellulose fibers, and a composite of said cotton fibers and biodegradable cellulose fibers;
wherein said wipe pad comprising a convex outer edge and a bow-shaped inner edge;
wherein said wipe pad comprising a pre-soaked wipe pad including said liquid solution for removing stains from oral surfaces when said wipe pad is rubbed against said oral surfaces;
wherein said oral surfaces selected from the group consisting of lips, teeth, and oral tissues;
wherein said wipe pad having an aperture at a first end originating from said inner edge for enabling said wipe pad to be used as a finger-sleeve; and
further wherein opposing sides of said wipe pad form said finger-sleeve to receive a fingertip of a user.

2. The oral care device of claim 1, wherein said wipe pad having a textured exterior surface for increasing friction for gentle scrubbing against said oral surfaces.

3. The oral care device of claim 1, wherein said wipe pad is a single use and disposable said wipe pad.

4. The oral care device of claim 1, wherein said liquid solution comprising vinegar, baking soda, soda water, water, and a natural flavoring agent selected from the group consisting of a spearmint oil, a peppermint oil, a citrus extract, and a menthol.

5. The oral care device of claim 4, wherein said vinegar having a % weight per volume of said liquid solution from 1% to 5%.

6. The oral care device of claim 5, wherein said baking soda having a % weight per volume of said liquid solution from 0.5% to 3%.

7. The oral care device of claim 6, wherein said natural flavoring agent having a % weight per volume of said liquid solution from 0.05% to 0.2%.

8. The oral care device of claim 7, wherein said soda water having a % weight per volume of said liquid solution from 0.1% to 0.5%.

9. The oral care device of claim 4, wherein said liquid solution further comprising a citric acid in an amount sufficient to adjust the pH to a value of from 4.5 to 5.5.

10. The oral care device of claim 1, wherein said aperture provides access to a tubular slit dimensioned to receive the fingertip of the user, and further wherein said wipe pad is flexible and conformable around the fingertip of the user.

11. The oral care device of claim 1, wherein said bow-shaped inner edge having a central dip and two peak members extending on opposing sides of said central dip.

12. An oral care device comprising:
a wipe pad; and
a liquid solution;
wherein said wipe pad comprising a convex outer edge and a bow-shaped inner edge;
wherein said wipe pad comprising a pre-soaked wipe pad including said liquid solution for removing stains from oral surfaces when said wipe pad is rubbed against said oral surfaces;
wherein said oral surfaces selected from the group consisting of lips, teeth, and oral tissues;
wherein said wipe pad having an aperture at a first end originating from said inner edge for enabling said wipe pad to be used as a finger-sleeve;
wherein opposing sides of said wipe pad form said finger-sleeve to receive a fingertip of a user; and
further wherein said liquid solution comprising vinegar, baking soda, soda water, water, and a natural flavoring agent selected from the group consisting of a spearmint oil, a peppermint oil, a citrus extract, and a menthol.

13. The oral care device of claim 12, wherein said vinegar having a % weight per volume of said liquid solution from 1% to 5%.

14. The oral care device of claim 12, wherein said baking soda having a % weight per volume of said liquid solution from 0.5% to 3%.

15. The oral care device of claim 12, wherein said natural flavoring agent having a % weight per volume of said liquid solution from 0.05% to 0.2%.

16. The oral care device of claim 12, wherein said soda water having a % weight per volume of said liquid solution from 0.1% to 0.5%.

17. The oral care device of claim 12, wherein said liquid solution further comprising a citric acid in an amount sufficient to adjust the pH to a value of from 4.5 to 5.5.

18. A method of forming and using an oral care device, the method comprising the steps of:
providing a wipe pad and a liquid solution;
wherein said wipe pad comprising a convex outer edge and a bow-shaped inner edge, wherein said wipe pad comprising a pre-soaked wipe pad including said liquid solution for removing stains from oral surfaces when said wipe pad is rubbed against said oral surfaces;
wherein said oral surfaces selected from the group consisting of lips, teeth, and oral tissues;
wherein said wipe pad having an aperture at a first end originating from said inner edge for enabling said wipe pad to be used as a finger-sleeve;
wherein opposing sides of said wipe pad form said finger-sleeve to receive a fingertip of a user;
wherein said liquid solution comprising a vinegar, a baking soda, a soda water, a water and a natural flavoring agent selected from the group consisting of a spearmint oil, a peppermint oil, a citrus extract, and a menthol;
sliding the fingertip of the user into said aperture and between said opposing sides of said wipe pad;
conforming said wipe pad around the fingertip of the user; and
rubbing said oral surfaces with said wipe pad.

19. The oral care device of claim 18, wherein said vinegar having a % weight per volume of said liquid solution from 1% to 5%; wherein said baking soda having a % weight per volume of said liquid solution from 0.5% to 3%; wherein said natural flavoring agent having a % weight per volume of said liquid solution from 0.05% to 0.2%; and further wherein said soda water having a % weight per volume of said liquid solution from 0.1% to 0.5%.

20. The oral care device of claim 18, wherein said liquid solution further comprising a citric acid in an amount sufficient to adjust the pH to a value of from 4.5 to 5.5.
